(19) United States
(12) Patent Application Publication
(10) Pub. No.: US 2006/0235523 A1
(43) Pub. Date: Oct. 19, 2006

(54) Implant having a sheath with a motion-limiting attribute

(75) Inventor: Carlos E. Gil, Collierville, TN (US)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 11/109,176

(22) Filed: Apr. 19, 2005

(51) Int. Cl. A61F 2/44

(52) U.S. Cl. 623/17.12; 623/23.41; 623/17.13

(57) ABSTRACT

A surgical implant containing two opposing shells, a central body disposed between the shells, and a flexible sheath extending between edges of the opposing shells. The sheath provides for resisting at least one predetermined type of relative directional motion.

### BACKGROUND

The present disclosure relates generally to prosthetic devices and systems and in particular to prosthetic devices and systems that provide spinal stabilization.

Spinal discs that extend between the endplates of adjacent vertebrae in a spinal column of the human body provide critical support between the adjacent vertebrae. These discs can rupture, degenerate and/or protrude by injury, degradation, disease or the like to such a degree that the intervertebral space between adjacent vertebrae collapses as the disc loses at least a part of its support function, which can cause impingement of the nerve roots and severe pain. In some cases, surgical correction may be required.

Typically, the surgical correction includes the removal of the spinal disc from between the adjacent vertebrae, and, in order to preserve the intervertebral disc space for proper spinal-column function, a prosthetic device is sometimes inserted between the adjacent vertebrae. In this context, prosthetic devices may be referred to as intervertebral prosthetic joints, prosthetic implants, disc prostheses or artificial discs, among other labels.

While preserving the intervertebral disc space for proper spinal-column function, most prosthetic devices permit at least one of the adjacent vertebrae to undergo different types of motion relative to the other, including bending and rotation. Bending may occur in several directions: flexion or forward bending, extension or backward bending, left-side bending (bending towards the human's left side), right-side bending (bending towards the human's right side), or any combination thereof. Rotation may occur in different directions: left rotation, that is, rotating towards the human's left side with the spinal column serving generally as an imaginary axis of rotation; and right rotation, that is, rotating towards the human's right side with the spinal column again serving generally as an imaginary axis of rotation.

In addition to the aforementioned motion types, some prosthetic devices further permit relative translation between the adjacent vertebrae in the anterior-posterior (front-to-back), posterior-anterior (back-to-front), medial-lateral right (middle-to-right side), or medial-lateral left (middle-to-left side) directions, or any combination thereof. Also, some prosthetic devices may permit combinations of the aforementioned types of motion.

### SUMMARY

The present disclosure relates generally to prosthetic devices and systems and in particular to prosthetic devices and systems that provide spinal stabilization.

According to one example, a device comprises a surgical implant. The surgical implant includes two opposing shells, a central body, and a sheath surrounding the shells and the central body. Each shell has an outer surface and an inner surface that is smoother than the outer surface. The outer surface is adapted to engage the surfaces of the bones of a joint in such a way that movement of the shell relative to the bone surface is resisted by friction between the outer surface and the surface of the bone.

The central body is disposed between the inner surfaces of the shells, and has an outer surface, at least a portion of which has a shape that complements and articulates with the shape of the inner surface of one or both of the shells.

The sheath is flexible and extends between edges of the opposing shells. The sheath has a motion-limiting attribute to resist movement outside a constrained range of motion, and an inner surface that, together with the inner surfaces of the shells, defines a cavity containing the central body.

According to another example, a system is provided that includes an implant adapted for insertion between adjacent vertebrae. The implant comprises two opposing shells, a central body, and means for encapsulating the central body between the opposing shells, which means also limits movement of the vertebrae adjacent to the implant to a constrained range.

According to another example, a method is provided that includes inserting an implant between adjacent vertebrae, and limiting movement at the site of implantation to a constrained range, which limiting of motion is caused by at least one component of the implant. According to one such method, the implant comprises two opposing shells, a central body, and a sheath. Each shell has an outer surface, an inner surface that is smoother than the outer surface, and an edge between the outer surface and the inner surface. The central body is disposed between the inner surfaces of the shells, and comprises an outer surface, at least a portion of which has a shape that complements and articulates with the shape of the inner surface of one or both opposing shells. The sheath extends between edges of the opposing shells, and has a motion-limiting attribute to limit movement of the shells to a constrained range. The sheath can provide for resisting at least one predetermined type of relative directional motion, and for allowing at least one other predetermined type of relative directional motion.

### BRIEF DESCRIPTION OF DRAWINGS

The disclosure can be more clearly understood by reference to the following drawings, which illustrate exemplary embodiments thereof, and which are not intended to limit the scope of the appended claims.

FIG. 1 is an exploded perspective view of an exemplary embodiment of an intervertebral endoprosthesis.

FIG. 2 is a sectional view of the intervertebral endoprosthesis shown in FIG. 1.

FIG. 3 is a perspective drawing of the intervertebral endoprosthesis shown in FIG. 1, assembled as a unitary structure.

FIG. 4 is an elevational view of the intervertebral endoprosthesis shown in FIG. 1.

FIG. 5 is a plan view of an implant plug and plug installation tool used to insert a plug into an intervertebral endoprosthesis.

FIG. 6 is a sectional view of the intervertebral endoprosthesis shown in FIG. 1, as implanted between two vertebrae.

The disclosure can be more clearly understood by reference to some of its specific embodiments, described in detail below, which description is not intended to limit the scope of the claims in any way.

### DETAILED DESCRIPTION OF SPECIFIC EMBODIMENTS

Implants as described herein can be used as a prosthetic implant in a wide variety of joints, including hips, knees, shoulders, etc. The description below focuses on an exemplary embodiment wherein the implant is a spinal disc endoprosthesis, but similar principles apply to adapt the implant for use in other joints. Those of skill in the art will readily appreciate that the particulars of the internal geometry will likely require modification from the description below to prepare an implant for use in other joints. However, the concept of using a sheath having a motion-limiting attribute and surrounding a core body disposed between opposing shells to provide relatively unconstrained movement of the respective surfaces until the allowable range of motion has been reached is applicable to use in any joint implant.

In broad aspect, the size and shape of the implant are substantially variable, and this variation will depend upon the joint geometry. Moreover, implants of a particular shape can be produced in a range of sizes, so that a surgeon can select the appropriate size prior to or during surgery, depending upon his assessment of the joint geometry of the patient, typically made by assessing the joint using CT, MRI, fluoroscopy, or other imaging techniques.

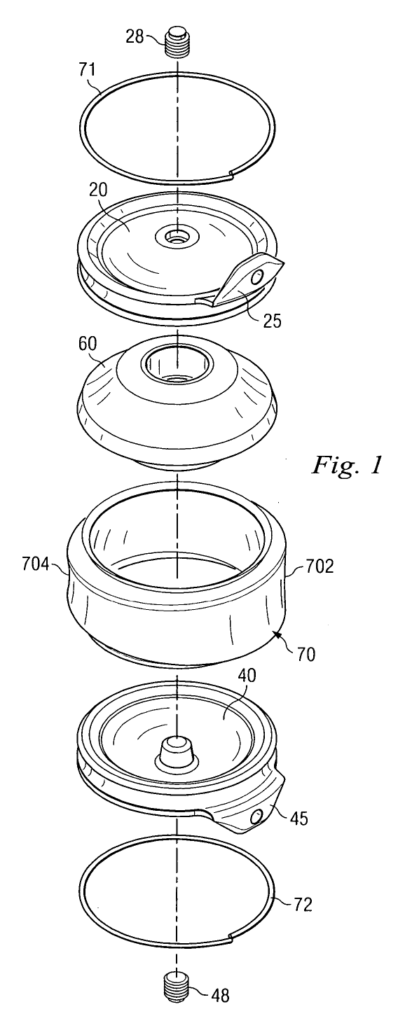

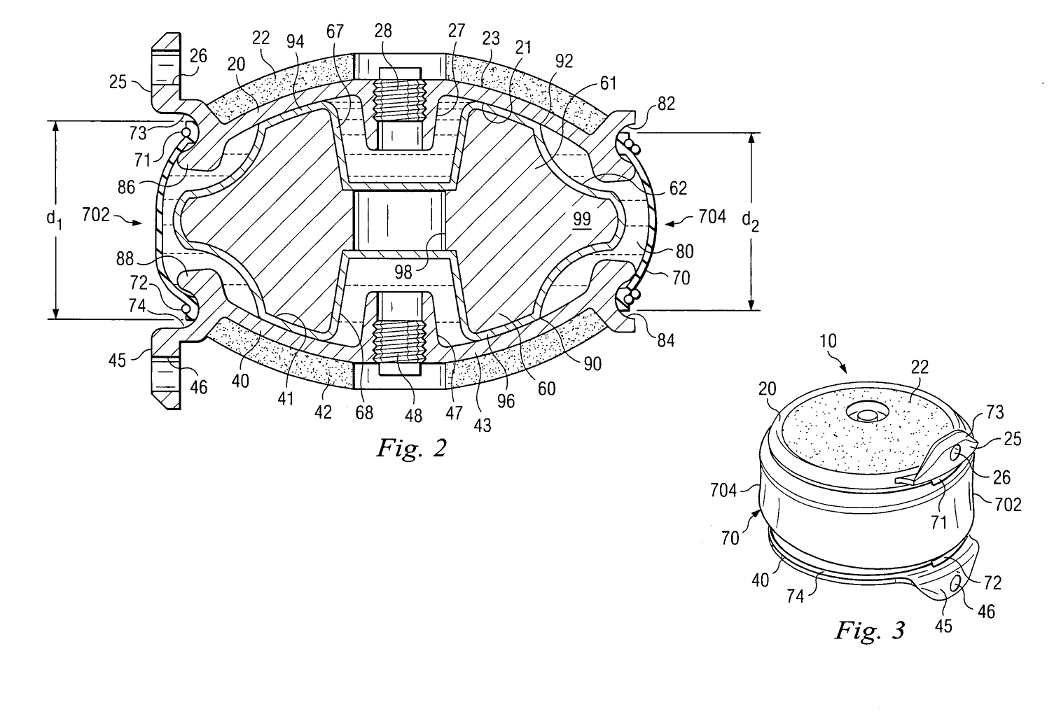

According to the exemplary embodiment illustrated in FIGS. 1 and 2, an implant comprises a first shell 20, a second shell 40, a central body 60, and a sheath 70.

Shells 20, 40 include outer convex surfaces 23, 43, and inner concave surfaces 21, 41. Outer convex surfaces 23, 43 are rough, in order to restrict motion of the shells relative to the bone surfaces that are in contact with the shells.

According to certain examples, the outer surfaces 23, 43 are coated with a biocompatible porous coating 22, 42. In certain examples, coating 22, 42 comprises a nonspherical sintered bead coating, while in other examples, coating 22, 42 comprises any coating that will promote bony ingrowth. A coating formed from nonspherical sintered beads provides for high friction between the outer surface of the shell and the bone, as well as providing an interaction with the cancellous bone of the joint, increasing the chances of bony ingrowth. One example of a suitable nonspherical sintered bead coating is that made of pure titanium, such as ASTM F-67. The coating can be formed by vacuum sintering.

At least a portion of the inner surface of each shell is smooth, and of a shape that complements and articulates with the shape of at least a portion of the central body. The inner surfaces of the shells are adapted to slide easily with low friction across a portion of the outer surface of the central body disposed between the shells. Desirably, the inner surfaces have an average roughness of about 1 to about 8 microinches, more particularly less than about 3 microinches. The central body has a shape that cooperates with the shape of the inner surface of the shell so as to provide motion similar to that provided by a healthy joint.

In certain examples, the shells, 20, 40 further include a number of geometric features that, as described in further detail below, cooperate with other components of the implant. Specifically, these features include a central retaining post 27, 47, an outer circumferential groove 82, 84, and radial stop 86, 88. The central retaining post 27, 47 extends axially from inner surfaces 21, 41. In addition, each shell 20, 40 includes an edge 73, 74, respectively. The outer circumferential grooves 82, 84 extend into the edges 73, 74 of the shells 20, 40. The radial stops 86, 88 extend from the edge 73, 74 in a direction generally perpendicular to the general plane of the shells 20, 40.

Radial stops 86, 88 and retaining posts 27, 47 help prevent the central body from being expelled from between the opposing shells when the shells are at maximum range of motion in flexion/extension. The hole receiving the post can have a diameter sufficiently large that relative motion between the shells and central body is unconstrained within an allowable range of motion, but that will nevertheless cause the post to arrest the central body before it is expelled from the implant under extreme compression. Alternatively, the diameter of the post may be such that it limits the translational movement of the central body during normal motion of the spine by contacting the surface of the hole in the central body at the limit of the allowable range of motion for the device.

Each shell may also be provided with tabs 25, 45. Tabs 25, 45 are optional features, but if present, extend from a portion of the edge 73, 74 in a direction generally perpendicular to the general plane of the shells 20, 40, and generally opposite the radial stops 86, 88. If present, tabs 25, 45 help to prevent long-term migration within the disc space, as well as catastrophic posterior expulsion, and the resulting damage to the spinal cord, other nerves, or vascular structures. Tabs 25, 45 may contain openings 26, 46 that can releasably engage an insertion tool (not shown).

The shells 20, 40, may be identical, or may be of different design (shape, size, and/or materials) to achieve different mechanical results. For example, differing plate or shell sizes may be used to more closely tailor the implant to a patient's anatomy, or to shift the center of rotation in the cephalad or caudal direction.

The shells can be made from any suitable biocompatible material. According to certain examples, the shells are made from a titanium alloy. In some such examples, the titanium alloy is ASTM F-136. In certain other examples, the shells are made of a biocompatible metal, such as stainless steel, cobalt chrome, or ceramics, such as those including Al₂O₃ or Zr₂O₃.

Central body 60 comprises a convex upper contact surface 94, a convex lower contact surface 96, and a central axial opening 98. In certain examples, central body member 60 includes an upper shoulder 92 and a lower shoulder 90. Each shoulder 90, 92 consists of an indentation in the surface of the central body member which defines a ledge that extends around the circumference of the central body 60. Shoulders 90, 92 can be used to constrain motion of the central body, and to provide a buffer that prevents contact between the shells. Preventing contact between the shells prevents friction and wear between the shells, thereby avoiding the production of particulates, which could cause increased wear on the internal surfaces of the implant.

The central body 60 is both deformable and resilient, and is composed of a material that has surface regions that are harder than the interior region. This allows the central body to be sufficiently deformable and resilient such that the implant functions effectively to provide resistance to compression and to provide dampening, while still providing adequate surface durability and wear resistance. In addition, the material of the central body has surfaces that are lubricious, in order to decrease friction between the central body and the opposing shells.

The material used to make the central body 60 is typically a slightly elastomeric biocompatible polymeric material. Examples of suitable polymeric materials include polyurethanes, such as poly carbonates and polyethers, polyurethane-containing elastomeric copolymers, such as polycarbonate-polyurethane elastomeric copolymers and polyether-polyurethane elastomeric copolymers. In certain examples, polyurethanes generally having a durometer hardness ranging from about 80 A to about 65 D (based upon raw, unmolded resin) are used.

In other examples, suitable polyurethanes include polycarbonates and polyethers, such as Chronothane P 75 A or P 55 D (P-eth-PU aromatic, CT Biomaterials); Chronoflex C 55D, C 65D, C 80A, or C 93A (PC-PU aromatic, CT Biomaterials); Elast-Eon II 80A (Si-PU aromatic, Elastomedic); Bionate 55D/S or 80A-80A/S (PC-PU aromatic with S-SME, PTG); CarboSil-10 90A (PC-Si-PU aromatic, PTG); Tecothane TT-1055D or TT-1065D (P-eth-PU aromatic, Thermedics); Tecoflex EG-93A (P-eth-PU aliphatic, Thermedics); and Carbothane PC 3585A or PC 3555D (PC-PU aliphatic, Thermedics).

The material used to make the central body 60 may be coated or impregnated to increase surface hardness, or lubricity, or both. Coating of the material used to form the central body may be done by any suitable technique, such as dip coating, and the coating solution may include one or more polymers, including those described above for the central body. The coating polymer may be the same as or different from the polymer used to form the central body, and may have a different durometer hardness from that used in the central body. Typical coating thickness is greater than about 1 mil, more particularly from about 2 mil to about 5 mil.

The central body 60 may also vary somewhat in shape, size, composition, and physical properties, depending upon the particular joint for which the implant is intended. The shape of the central body should complement that of the inner surface of the shell to allow for a range of translational, flexural, extensional, and rotational motion, and lateral bending appropriate to the particular joint being replaced.

Attachment of the sheath 70 to the shells 20, 40 can be accomplished in a variety of ways. According to one example, attachment of the sheath 70 to the shells 20, 40 comprises providing the edge of each shell with a circumferential groove (the term “circumferential” in this context does not imply any particular geometry).

The sheath 70 can be disposed so that the edges of the sheath 70 overlap the outer circumferential grooves 82, 84 of the shells 20, 40. Retaining rings 71, 72 are then placed over the edges of the sheath 70 and into the circumferential grooves 82, 84, thereby holding the flexible sheath in place and attaching it to the shells. The retaining ring can be formed by wrapping a wire around the groove over the overlapping portion of the sheath, cutting the wire to the appropriate size, and welding the ends of the wire to form a ring.

While any suitable biocompatible material can be used for the retaining rings, stainless steel, titanium or titanium alloys are particularly suitable. The retaining rings are desirably fixed in place by, e.g., welding the areas of overlap between the ends of the retaining rings. Because of the high temperatures needed to weld titanium and titanium alloys, and because of the proximity of the weld area to both the sheath 70 and the central body 60, laser welding is typically used.

Sheath 70 is made from a flexible material. According to one example, the sheath is made from a biocompatible elastomeric polymeric material, such as segmented polyurethane, or polyethylene. Other examples of suitable polymeric materials for forming the sheath 70 include polyurethanes, such as poly carbonates and polyethers, polyurethane-containing elastomeric copolymers, such as polycarbonate-polyurethane elastomeric copolymers and polyether-polyurethane elastomeric copolymers. In certain examples, polyurethanes generally having a durometer hardness ranging from about 80 A to about 65 D (based upon raw, unmolded resin) are used. In still other examples, suitable materials for forming sheath 70 include materials commercially known as BIOSPAN-S (aromatic polyetherurethaneurea with surface modified end groups, Polymer Technology Group), CHRONOFLEX AR/LT (aromatic polycarbonate polyurethane with low-tack properties, CardioTech International), CHRONOTHANE B (aromatic polyether polyurethane, CardioTech International), CARBOTHANE PC (aliphatic polycarbonate polyurethane, Thermedics). In still other examples, the flexible material comprising the sheath may be reinforced with fibers of polyethylene, polyglycolic acid, polytetrafluroethylene, or polyester.

In certain examples, the thickness of the sheath is in the range of from about 5 to about 30 mils, and in other examples, about 10-11 mils.

The sheath 70 has a motion-limiting attribute that limits the range of motion allowed at the site where the implant is inserted. Limiting the range of motion can include resisting at least one predetermined type of relative directional motion, for example, motion in an anterior direction, and allowing at least one other predetermined type of relative directional motion, for example, motion in a posterior direction.

According to certain examples, the motion-limiting attribute comprises a trapezoidal configuration such that the anterior aspect of the sheath 702 is greater in height than the posterior aspect of the sheath 704. The greater height of the anterior aspect 702 of the sheath 70 relative to the posterior aspect 704 of the sheath limits at least one of anterior-posterior flexion, anterior-posterior extension and anterior-posterior translation. According to such examples, the anterior aspect 702 of the sheath 70 will limit the range of motion in an anterior direction, relative to the posterior direction, while permitting a greater range of motion in the posterior direction, relative to the anterior direction. Sheath 70 can also help prevent the central body from being expelled from between the opposing shells, in a manner similar to that of radial stops 86, 88 and retaining posts 27, 47.

Other components of the implant, for example the central body 60, and shells 20, 40, can provide features that contribute to the limitation of motion. As discussed above, radial stops on the shells and shoulders on the central body can be used to constrain motion. For example, contact of the walls or extensions 86, 88 of the shells with shoulders 90, 92 of the central body may also contribute to limiting the range of motion to that desired. The central retaining posts 27, 47 may also contribute to limiting the range of motion by contact with the central axial opening of the central body.

In some examples, limitation of motion provided by the shells and/or the central body can be in addition to the limitation of motion provided by the sheath. In other examples, such function of the shells and/or the central body can be a replacement for the limitation of motion provided by the sheath, for example, when the sheath is at a maximum range of motion that it can constrain, features of the shells and/or central body can take over at such range. In still other examples, such function of the shells and/or central body can provide for limitation of motion in a direction other than that provided by the sheath.

Thus, in certain examples, the kinematics of the motion provided by the implant are defined primarily by the sheath, the central body 60, and the shells 20, 40. Although the central body is encapsulated within the sheath and the shells, it is not attached to these components. Accordingly, the central body 60 freely moves within the enclosed structure provided by the sheath 70 and shells 20, 40, but is constrained by limitations imposed by the sheath 70, and, if used, geometric limitations imposed by interaction between the shells and the central body.

An example of a geometry of the sheath, shells and central body that limits the motion of the central body is illustrated in FIG. 2. The anterior aspect 702 of the sheath is greater in height, as illustrated by d₁ than the posterior aspect 704, as illustrated by d₂. The respective heights d₁ and d₂ of the respective anterior and posterior aspects of the sheath result in those components of the sheath in proximity to the anterior portion being more spaced apart than those components of the sheath in proximity to the posterior portion. The respective heights d₁ and d₂ of the anterior and posterior aspects also provide the sheath with a trapezoidal configuration. In certain examples, this trapezoidal configuration limits the range of motion at the implant site in an anterior direction, for example, at least one of translation, flexion and extension in an anterior direction. In certain examples, when this motion-limiting attribute of the sheath has reached the maximum range of motion it can constrain, other features of the implant, such as the shells and the central body, can provide further or additional restraint.

For example, extensions 86, 88 on shells 20, 40 can contact shoulders 90, 92 on the central body 60. Specifically, the inner portion of the extension 86, 88 forms a circumferential ridge that limits the range of motion of the shells 20, 40 relative to the central body 60 by contacting central body shoulders 90, 92. This limitation of motion can occur during or subsequent to the limitation of motion provided by the sheath.

As explained above, in one embodiment, the shells are concavo-convex, and their inner surfaces mated and articulated with a convex outer surface of the central body. The sheath is secured to the rims of the shells with retaining rings, and which, together with the inner surfaces of the shells, forms an implant cavity. In a particular aspect of this embodiment, using a coordinate system wherein the geometrical center of the implant is located at the origin, and assigning the x-axis to the anterior (positive) and posterior (negative) aspect of the implant, the y-axis to the right (positive) and left (negative) aspect of the implant, and the z-axis to the cephalad (positive) and caudal (negative) aspects of the implant, the convex portion of the outer surface and the concave portion of the inner surface of the shells can be described as quadric surfaces, such that x²/a²+y²/b²+z²/c²=1, where (+/−a,0,0), (0,+/−,0), and (0,0,+/−c) represent the x, y, and z intercepts of the surfaces, respectively. Typical magnitudes for a, b, and c are about 11 mm, 30 mm, and 10 mm, respectively.

The implant is symmetrical about the x-y plane, and is intended to be implanted in the right-left center of the disc space, but may or may not be centered in the anterior-posterior direction. In any event, the implant is not allowed to protrude in the posterior direction past the posterior margin of the vertebral body.

In the coordinate system described above, the central axis of retaining post 27, 47 is typically coincident with the z-axis, but may move slightly to accommodate various clinical scenarios. The shape of the post may be any quadric surface. However, a truncated tapered elliptical cone is a particularly suitable geometry. Similarly, the geometry of the central axial opening of the central body will correspond to the geometry of the retaining post, and will have a similar geometry.

The central body contains surfaces that are described by an equation similar to that for the inner surfaces of the shells, and which articulates with those inner surfaces. The central body will have a plane of symmetry if identical opposing shells are used.

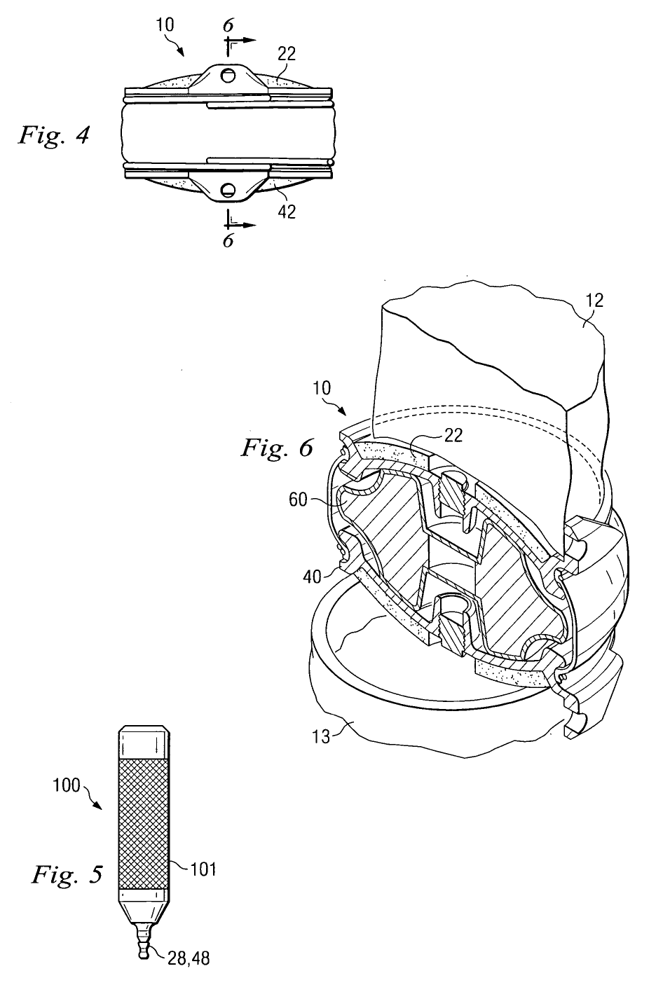

The complete assembly of the exemplary implant illustrated in FIG. 1 is illustrated in FIGS. 3 and 4, wherein the central body 60 is bracketed between shells 20, 40. The flexible sheath 70 extends between the two opposing shells 20, 40, and encapsulates the central body 60 such that the implant is a unitary structure. FIG. 6 illustrates the implant inserted as a unitary structure between two vertebrae.

According to certain embodiments, means for accessing the interior of the implant after it has been assembled into a unitary structure are provided. This means consists of a central axial opening included in the shells 20, 40. Typically, this opening will be provided through central retaining posts 27, 47. By providing access to the interior of the implant, sterilization can be done just prior to implantation. Sterilization is preferably accomplished by introducing an ethylene oxide surface sterilant. Caution should be exercised in using irradiation sterilization, as this can result in degradation of the polymeric materials in the sheath or central body, particularly if these include polyurethanes.

After sterilization, the central openings can be sealed using plugs 28, 48. Preferably, only one plug is inserted first. The plug is inserted using insertion tool 100, shown in FIG. 5, and which contains handle 101 and detachable integral plug 28, 48. The tool is designed so that plug 28, 48 detaches from the tool when a predetermined torque has been reached during insertion of the plug. The tool can then be discarded.

After one plug has been inserted to one of the shells, a lubricant 80 is preferably introduced into the interior of the device prior to inserting the second plug. To do this a syringe is used to introduce the lubricant into the remaining central opening, and the implant is slightly compressed to remove some of the excess air. Another insertion tool 100 is then used to insert a plug into that central opening, and thereby completely seal the interior of the device from its exterior environment. In certain examples, the lubricant 80 is saline. In other examples, other lubricants may be used, for example, hyaluronic acid, mineral oil, and the like.

Where the implant is used as an endoprosthesis inserted between two adjacent vertebral bodies, the implant may be introduced using a posterior or anterior approach. For cervical implantation, an anterior approach is preferred. The implanting procedure is carried out after discectomy, as an alternative to spinal fusion. The appropriate size of the implant for a particular patient, determination of the appropriate location of the implant in the intervertebral space, and implantation are all desirably accomplished using precision stereotactic techniques, apparatus, and procedures, such as the techniques and procedures known to those of ordinary skill in the art. Non-stereotactic techniques can also be used. In either case, discectomy is used to remove degenerated, diseased disc material and to provide access to the intervertebral space sufficient to prepare the surfaces of the vertebral bodies for insertion of the implant. To prepare the vertebral bodies, a cutting or milling device is used to shape the endplates of the vertebral bodies to complement the outer surfaces of the implant and to expose cancellous bone.

This access is used to remove a portion of the vertebral body using a burr or other appropriate instruments, in order to provide access to the intervertebral space for a transverse milling device. Transverse milling devices, and use and acquisition thereof, are known to those of ordinary skill in the art. The milling device is used to mill the surfaces of the superior and inferior vertebral bodies that partially define the intervertebral space to create an insertion cavity having surfaces that (a) complement the outer surfaces of the implant and (b) contain exposed cancellous bone.

This provides for an appropriate fit of the implant with limited motion during the acute phase of implantation, thereby limiting the opportunity for fibrous tissue formation, and increases the likelihood for bony ingrowth, thereby increasing long-term stability.

The invention has been described above with respect to certain specific embodiments thereof. Those of skill in the art will understand that variations from these specific embodiments that ate within the spirit of the invention will fall within the scope of the appended claims and equivalents thereto.

What is claimed is:

1. A surgical implant comprising:
two opposing shells, each having
an outer surface adapted to engage the surfaces of the bones of a joint in such a way that movement of the shell relative to the bone surface is resisted by friction between the outer surface and the surface of the bone;
an inner surface that is smoother than the outer surface; and
an edge between the outer surface and the inner surface;
a central body disposed between the inner surfaces of the shells comprising an outer surface, at least a portion of which has a shape that complements and articulates with the shape of the inner surface of one or both opposing shells; and
a sheath extending between edges of the opposing shells, having a motion-limiting attribute to resist movement outside a constrained range of motion, and an inner surface that, together with the inner surfaces of the shells, defines a cavity containing the central body.

2. The surgical implant of claim 1 wherein the motion-limiting attribute comprises an anterior aspect of the sheath that is greater in height than the posterior aspect of the sheath.

3. The surgical implant of claim 1 wherein the sheath is made from an elastomeric polymeric material.

4. The surgical implant of claim 3 wherein the elastomeric polymeric material is selected from the group consisting of polyurethane, polyethylene, poly carbonates and polyethers.

5. The surgical implant of claim 3 wherein the elastomeric polymeric material is a copolymer selected from the group consisting of polyurethane-containing elastomeric copolymers and polyether-polyurethane elastomeric copolymers.

6. The surgical implant of claim 1, further comprising:
a liquid lubricant, which occupies at least a portion of the cavity.

7. The surgical implant of claim 1 further comprising:
a motion-limiting device disposed on the inner surface of at least one of the opposing shells.

8. The surgical implant of claim 7, wherein the motion limiting device comprises an extension formed on the inner surface.

9. The surgical implant of claim 8, wherein the extension is located at the edge of the shell, and extends toward the central body.

10. The surgical implant of claim 7, wherein the surface of the central body comprises a motion limiting device disposed thereon, which contacts the motion limiting device of the shell when the implant reaches the end of an acceptable range of motion.

11. The surgical implant of claim 10, wherein the motion limiting device on the central body comprises a shoulder.

12. The surgical implant of claim 7, wherein the motion limiting device comprises a post extending toward the central body, and wherein the outer surface of the central body further comprises at least one opening adapted to receive the post.

13. The surgical implant of claim 1, wherein the edge of at least one of the opposing shells comprises a tab extending axially away from the central body.

14. The surgical implant of claim 13, wherein the tab is adapted to releasably receive a tool for manipulating, inserting or removing the implant.

15. The surgical implant of claim 14, wherein the edges of both opposing shells comprise a tab.

16. The surgical implant of claim 1, wherein the outer surface of each opposing shell is coated with a biocompatible porous coating.

17. The surgical implant of claim 1 wherein at least one of the opposing shells further comprises a closable passage between its outer surface and its inner surface.

18. The surgical implant of claim 17, wherein the closable passage comprises a hole that is closable by insertion of a correspondingly sized plug.

19. The surgical implant of claim 1 wherein the edge between the outer surface and the inner surface of the rigid opposing shells comprises a circumferential groove adapted to receive a retaining ring.

20. The surgical implant of claim 19, wherein the sheath overlaps the circumferential groove and is held against the edge of the opposing shells by the retaining ring.

21. A system comprising an implant adapted for insertion between adjacent vertebrae, which implant comprises two opposing shells, a central body, and means for encapsulating the central body between the opposing shells, which means also limits movement of the vertebrae adjacent to the implant to a constrained range.

22. The system of claim 19 wherein the means limits at least one of anterior-posterior flexion, anterior-posterior extension and anterior-posterior translation.

23. A method comprising:
inserting an implant between adjacent vertebrae, which implant comprises two opposing shells, each shell having an outer surface, an inner surface that is smoother than the outer surface; and an edge between the outer surface and the inner surface; a central body disposed between the inner surfaces of the shells, the central body comprising an outer surface, at least a portion of which has a shape that complements and articulates with the shape of the inner surface of one or both opposing shells; and a sheath extending between edges of the opposing shells, having a motion-limiting attribute; and
limiting movement of the vertebrae adjacent to the implant to a constrained range, which limiting of motion is caused at least in part by the motion-limiting attribute of the flexible sheath.

24. The method of claim 23 wherein the motion-limiting attribute comprises an anterior aspect of the sheath that is greater in height than the posterior aspect of the sheath.

25. The method of claim 23 wherein the motion-limiting attribute limits at least one of anterior-posterior flexion, anterior-posterior extension and anterior-posterior translation.
